SEQUENCE LISTING (2 sequences)

SEQ ID NO: 1 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 2 - protein, 660 aa, Homo sapiens
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

(19) United States
(12) Patent Application Publication
(10) Pub. No.: US 2023/0305010 A1
(43) Pub. Date: Sep. 28, 2023

(54) TIMP1 AS A MARKER FOR CHOLANGIOCARCINOMA

(71) Applicant: Roche Diagnostics Operations, Inc., Indianapolis, IN (US)

(72) Inventors: Farshid Dayyani, Irvine, CA (US); Anika Mang, Holzkirchen (DE); David Morgenstern, Indianapolis, IN (US); Vinzent Rolny, München (DE); Magdalena Swiatek-De Lange, Penzberg (DE)

(21) Appl. No.: 18/309,985

(22) Filed: May 1, 2023

Related U.S. Application Data
(63) Continuation of application No. PCT/EP2020/079508, filed on Oct. 30, 2020.

(51) Int. Cl. G01N 33/574

(52) U.S. Cl. CPC G01N 33/57438; G01N 2333/8146; G01N 2333/96494; G01N 2470/04; G01N 2800/50; G01N 2800/08

(57) ABSTRACT

The present invention relates to an in vitro method for assessing cholangiocarcinoma in a patient sample, comprising the steps of: a) determining the level of tissue inhibitor of metalloproteinase-1 (TIMP1) in the patient sample, wherein the patient sample is selected from a group consisting of serum, plasma and whole blood sample from an individual, b) comparing the level of TIMP1 determined in step (a) with a reference level of TIMP1, and c) assessing cholangiocarcinoma in the patient sample by comparing the level determined in step (a) to the reference level of TIMP1, wherein an increased level of TIMP1 compared to the reference level of TIMP1 is indicative for cholangiocarcinoma in the patient sample. Further, the present invention relates to an in vitro method for assessing cholangiocarcinoma comprising TIMP1 and MMP2, the use of TIMP1 and optionally MMP2 in the in vitro assessment of CCA, and a kit for performing the said methods.

### FIELD OF THE INVENTION

The present invention relates to in vitro methods for assessing cholangiocarcinoma in a patient sample and a kit for performing the said methods. Further, the present invention relates to the use of TIMP1 as a marker molecule and a marker combination comprising TIMP1 and MMP2 in the in vitro assessment of cholangiocarcinoma, respectively.

### BACKGROUND OF THE INVENTION

Cholangiocarcinoma (abbreviated as CCA or CCC) is a highly fatal malignant biliary tract tumor arising from the epithelium of the bile duct. CCA accounts for about 3% of all gastrointestinal tumors and is the second most common primary liver tumor after hepatocellular carcinoma (abbreviated as HCC). On the basis of its anatomical origin, CCA is classified as intrahepatic (abbreviated as iCCA), perihilar (abbreviated as pCCA), or distal (abbreviated as dCCA) CCA (Rimola J. et al., Hepatology, 2009, 50 (3):791-798). The incidence of CCA, particularly iCCA, appears to be increasing and may be as high as 2.1 per 100,000 person years in Western Countries, with highest known rates in Northeast Thailand (>80 per 100,000; Martha M. Kirstein, Arndt Vogel, Epidemiology and Risk Factors of Cholangiocarcinoma, Visc Med 2016; 32:395-400).

CCA has similar risk factors to HCC, including cirrhosis, chronic viral hepatitis, alcohol excess, diabetes, and obesity. Further well established risk factors for CCA are associated with chronic biliary inflammation, e.g. hepatobiliary flukes, primary sclerosing cholangitis (abbreviated as PSC), biliary tract cysts, hepatolithiasis and toxins (Bridgewater J. et al., J Hepatol, 2014, 6:1268-893, 8-14; Khan S. A. et al., Consensus document. Gut., 2002, 51 Suppl 6:VI1-9; Khan S. A. et al., Gut. 2012, 61(12):1657-1669; Alvaro D et al., Dig Liver Dis., 2010, 42:831-838; Benavides M. et al., Clin Transl Oncol., 2015, 17:982-987; Benson III A. B. et al., J Natl Compr Cane Netw., 2009, 7:350-391; Cai J. Q. et al., J Huazhong Univ Sci Technolog Med Sci., 2014, 34:469-475; Rerknimitr R. et al., J Gastroenterol Hepatol., 2013, 28:593-607).

CCA diagnosis is often complex and requires the use of multiple diagnostic modalities to distinguish between benign and malignant structures, to differentiate CCA from other primary liver tumors, mainly HCC and combined HCC-CCAs and to stage and grade the tumors. Differentiation between CCA, in particular iCCA, and HCC is essential for surgical planning and prognosis assessment. Imaging methods, like dynamic computer tomography scans, can differentiate between HCC and CCA based on heterogeneous contrast uptake and absence of contrast washout in delayed phases characteristic for cholangiocarcinoma (Rimola J. et al., Hepatology 2009, 50 (3):791-798). However, these classic features of intrahepatic cholangiocarcinoma are not universally present in all CCA cases (lavarone M. et al., J Hepatol., 2013, 58:1188-93; Kim S. H. et al., J Comput Assist Tomogr., 2012, 36:704-09).

To date, there is no specific blood test available for specific diagnose of CCA. Carbohydrate antigens CA19-9 and CA-125, and carcinoembryonic antigen CEA are most widely used markers for suspected CCA, but their diagnostic performance is very limited. CA19-9 is elevated in pancreatic, colorectal, and gastric cancers, as well as in nonmalignant conditions, such as PSC (Primary Sclerosing Cholangitis) and obstructive jaundice. Additionally, patients lacking Lewis antigen (10% of the general population) cannot produce CA19-9 and thus do not benefit from testing. The sensitivity and specificity of CA19-9 in CCA patients range from 40% to 70% and 50% to 80%, respectively, with a positive predictive value of 16%-40%. CEA is raised in 20-30% of patients with CCA, whereas CA-125 is elevated in approx. 40%-50%, what's insufficient for accurate diagnosis (Alsaleh M. et al., Int J Gen Med., 2018, 12:13-23; Khan S. A. et al., Gut., 2002, 51 Suppl 6:VI1-9; Patel A. H. et al., Am J Gastroenterol, 2000, 95:204-207; Hultcrantz R. et al., J Hepatol, 1999, 30:669-673). Those limitations are recognized by major international guidelines, recommending CEA and CA19-9 measurement only as a supportive, but not diagnostic tool in cholangiocarcinoma management (Khan S. A. et al., Consensus document. Gut., 2002, 51 Suppl 6:VI1-9; Khan S. A. et al., Gut. 2012, 61(12):1657-1669; Alvaro D. et al., Dig Liver Dis., 2010, 42:831-838; Benavides M. et al., Clin Transl Oncol., 2015, 17:982-987; Benson III A. B. et al., J Natl Compr Canc Netw., 2009, 7:350-391; Cai J. Q. et al., J Huazhong Univ Sci Technolog Med Sci., 2014, 34:469-475; Rerknimitr R. et al., J Gastroenterol Hepatol., 2013, 28:593-607).

Therefore, identification of novel biomarkers for more specific diagnosis and stratification of CCA is recognized as one of unmet medical needs by current EASL guidelines for the diagnosis and management of intrahepatic cholangiocarcinoma (Bridgewater J., J Hepatol., 2014, 6:1268-89).

As described above, all current methods for diagnosis and differential diagnosis of cholangiocarcinoma have major shortcomings:

Ultrasound: ultrasonography (abbreviated as US) is reliable for excluding gallstones but is operator-dependent and is insufficient alone for investigating suspected CCA. US may miss small tumors and cannot accurately define tumor extent (Khan S. A. et al., Gut., 2002, 51 Suppl 6:VI 1-9; Khan S. A. et al., Gut. 2012, 61(12):1657-1669). Only the Japanese guideline and the 2002 edition of the BSG guideline recommended US for initial examination included in diagnostic algorithms (Khan S. A. et al., Gut., 2002, 51 Suppl 6:VI 1-9; Khan S. A. et al., Gut., 2012, 61(12):1657-1669).

Contrast-enhanced ultrasound (abbreviated as CEUS) improves the diagnostic performance significantly in the differentiation between intraheptic cholangiocarcinoma (iCCA) and HCC compared to baseline ultrasound, but its performance is strongly reader-dependent, with AUC varying from 0.650 to 0.933, as reported in Chen L. D. et al., Eur Radiol., 2010, 20(3):743-53. Additionally, the usefulness of CEUS in diagnosing small ICCs is not known.

High resolution/spiral computer tomography (abbreviated as CT): Contrast CT has higher sensitivity for CCA detection than US (up to 80%), providing good views of intrahepatic mass lesions, dilated intrahepatic ducts, localized lymphadenopathy and extrahepatic metastases. However, unlike HCC (Sun H. and Song T., Drug Discov Ther., 2015, 9:310-318), the radiological criteria of CT or magnetic resonance imaging (abbreviated as MRI) are insensitive for the diagnosis of CCA. Thus, pathological diagnosis is required for a definitive diagnosis of CCA. Moreover, CT/MRI may miss small lesions (Chen L. D. et al., Eur Radiol., 2010, 20(3):743-53; Hanninen E. L. et al., Acta Racliol., 2005, 46:462-470).

Serum tumor markers: Carbohydrate antigens CA19-9 and CA-125 and carcinoembryogenic antigen CEA are the most used serum tumor markers. All of them have significant overlap with other benign diseases and low sensitivity for early stage disease, which limits their use for diagnosis. The sensitivity and specificity of CA 19-9 for iCCA is only 62% and 63%, respectively. Diagnostic performance of CEA is even lower, as it is elevated in only 20-30% of CCA patients, whereas CA-125 is elevated in approximately 40%-50%, what is insufficient for accurate diagnosis (Alsaleh M. et al., Int J Gen Med., 2018, 12:13-23; Khan S. A. et al., Gut., 2002, 51 Suppl 6:VI1-9; Patel A. H. et al., Am J Gastroenterol, 2000, 95:204-207; Flultcrantz R. et al J Hepatol, 1999, 30:669-673).

Whole blood, serum or plasma are the most widely used sources of patient sample in clinical routine. The identification of an early CCA marker that would aid in the reliable CCA detection or provide early prognostic information could lead to a method that would greatly aid in the diagnosis and in the management of this disease.

Therefore, an urgent clinical need exists to improve the in vitro assessment of CCA. It is especially important to improve the early diagnosis of CCA, since for patients diagnosed in early stages of CCA the chances of reversibility of bile duct damages are much higher as compared to those patients diagnosed at a more progressed stage of disease.

There is a strong need in the art to identify a reliable and straightforward indicator of the CCA disease state both in order to reliably distinguish the symptoms of CCA from those of the above mentioned other respiratory diseases, to predict changes in disease severity, disease progression and response to medicine.

It is an object of the present invention to provide a simple and cost-efficient procedure of CCA assessments, e.g. to identify individuals suspected of having CCA. In particular, an object of the present invention is to provide in vitro methods for assessing cholangiocarcinoma in a patient sample and a kit for performing the said methods. Further, an object of the present invention relates to the use of TIMP1 as a marker molecule and a marker combination comprising TIMP1 and MMP2 in the in vitro assessment of cholangiocarcinoma, respectively.

This object or these objects is/are solved by the subject matter of the independent claims. Further embodiments are subjected to the dependent claims.

### SUMMARY OF THE INVENTION

It has now been found that the use of tissue inhibitor of metalloproteinase-1 (TIMP1) can at least partially overcome some of the problems of the methods available for assessment of CCA presently known.

Surprisingly it was found in the present invention that an in vitro method comprising the step of determining the level of TIMP1 in a patient sample allows for the assessment of CCA. In this context it was found that an elevated level of said TIMP1 in such sample obtained from an individual compared to a reference level for TIMP1 is indicative for the presence of CCA.

In a first aspect, the present invention relates to an in vitro method for assessing cholangiocarcinoma in a patient sample, comprising:
a) determining the level of tissue inhibitor of metalloproteinase-1 (TIMP1) in the patient sample, wherein the patient sample is selected from a group consisting of serum, plasma and whole blood sample from an individual,
b) comparing the level of TIMP1 determined in step (a) with a reference level of TIMP1, and
c) assessing cholangiocarcinoma in the patient sample by comparing the level determined in step (a) to the reference level of TIMP1, wherein an increased level of TIMP1 compared to the reference level of TIMP1 is indicative for cholangiocarcinoma in the patient sample.

In a second aspect, the present invention relates to the use of TIMP1 as a marker molecule in the in vitro assessment of cholangiocarcinoma in a serum, plasma or whole blood sample of an individual, wherein a level of TIMP1 above a reference level of TIMP1 is indicative for cholangiocarcinoma.

In a third aspect, the present invention relates to the use of a marker combination comprising TIMP1 and MMP2 in the in vitro assessment of cholangiocarcinoma in a serum, plasma or whole blood sample of an individual, wherein the levels of TIMP1 and MMP2 are indicative for cholangiocarcinoma. According to the third aspect of the present invention, the levels of TIPM1 and MMP2 can in particular mean the detected levels of TIMP1 and MMP2 or the detection of TIMP1 and MMP2.

In a fourth aspect, the present invention relates to an in vitro method for assessing cholangiocarcinoma in a patient sample, comprising:
(a′) determining the level of tissue inhibitor of metalloproteinase-1 (TIMP1) in the patient sample, wherein the patient sample is selected from a group consisting of serum, plasma and whole blood sample from an individual,
(b′) determining the level of matrix metalloproteinase-2 (MMP2) for cholangiocarcinoma in the patient sample, and
(c′) assessing cholangiocarcinoma by comparing the determining results of steps (a′) and (b′), wherein the levels of TIMP1 and MMP2 are indicative for CCA. According to the fourth aspect of the present invention, the levels of TIPM1 and MMP2 can in particular mean the detected levels of TIMP1 and MMP2 or the detection of TIMP1 and MMP2.

In a fifth aspect, the present invention relates to a kit for performing at least one of the said methods comprising reagents, which are required to determine the level of TIMP1 determined in step (a) or step (a′) and optionally to determine the level of MMP2 determined in step (b′).

### LIST OF FIGURES

FIG. 1 shows the boxplot distribution of the determined TIMP1 level values (concentration values in ng/ml) according to CCA (CCC) of 55 samples, HCC of 219 HCC samples and at-risk controls of 632 samples.

FIG. 2 shows the plot of the receiver operator characteristics (ROC-plot, univariate analysis) of CCA vs. at-risk controls samples with an AUC of 0.939 and the plot of the receiver operator characteristics (ROC-plot) of CCA vs. HCC samples with an AUC of 0.715; X-axis: 1−specificity (false positive); Y-axis: sensitivity (true positive).

FIG. 3 shows the boxplot distribution of the determined TIMP1 level values (concentration values in ng/ml) according to CCA (CCC) of 55 samples and HCC and at-risk controls of 851 samples.

FIG. 4 shows the plot of the receiver operator characteristics (ROC-plot, univariate analysis) of TIMP1 in differentiating CCA from HCC+at-risk controls with an AUC of 0.881 X-axis: 1−specificity (false positive); Y-axis: sensitivity (true positive).

FIG. 5 shows the boxplot distribution of the of the multivariate score of the marker combination TIMP1 and MMP2 according to CCA (CCC) of 55 samples and HCC of 219 samples.

FIG. 6 shows the plot of the receiver operator characteristics (ROC-plot, multivariate analysis) of the marker combination TIMP1 and MMP2 in differentiating CCA from HCC with an AUC of 0.922; X-axis: 1−specificity (false positive); Y-axis: sensitivity (true positive).

FIG. 7 shows the boxplot distribution of the multivariate score of the marker combination TIMP1 and MMP2 according to CCA (CCC) of 55 samples and at-risk controls of 632 samples.

FIG. 8 shows the plot of the receiver operator characteristics (ROC-plot, multivariate analysis) of the marker combination TIMP1 and MMP2 in differentiating CCA from at-risk controls with an AUC of 0.977; X-axis: 1−specificity (false positive); Y-axis: sensitivity (true positive).

FIG. 9 shows the boxplot distribution of the multivariate score of the marker combination TIMP1 and MMP2 according to CCA (CCC) of 55 samples and HCC+at-risk controls of 851 samples.

FIG. 10 shows the plot of the receiver operator characteristics (ROC-plot, multivariate analysis) of the marker combination TIMP1 and MMP2 in differentiating CCA vs. HCC+at-risk controls with an AUC of 0.957; X-axis: 1−specificity (false positive); Y-axis: sensitivity (true positive).

### DETAILED DESCRIPTION OF THE INVENTION

Before the present invention is described in detail below, it is to be understood that this invention is not limited to the particular embodiments and examples described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

The term “in vitro method” is used to indicate that the method is performed outside a living organism and preferably on body fluids, isolated tissues, organs or cells.

The term “assessing cholangiocarcinoma (CCA)” is used to indicate that the method according to the present invention will aid a medical professional including, e.g., a physician in assessing whether an individual has disease CCA or is at risk of developing disease CCA.

A “level of TIMP1 above the reference level” indicates that the individual has disease CCA or that the individual is at risk of developing disease CCA or prognosing the course of disease CCA. The term “reference level” or reference sample as used herein, refers to a a sample or a level of a sample which is analysed in a substantially identical manner as the sample or the level of the sample of interest and whose information is compared to that of the sample of interest. A reference level or sample thereby provides a standard allowing for the evaluation of the information obtained from the sample of interest. A reference level or reference sample may be derived from a healthy or normal tissue, organ or individual, thereby providing a standard of a healthy status of a tissue, organ or individual. Differences between the status of the normal reference sample or normal reference level and the status of the sample of interest may be indicative of the risk of disease development or the presence or further progression of such disease or disorder. A reference sample or reference level may be derived from an abnormal or diseased tissue, organ or individual thereby providing a standard of a diseased status of a tissue, organ or individual. Differences between the status of the abnormal reference sample or abnormal reference level and the status of the sample of interest may be indicative of a lowered risk of disease development or the absence or bettering of such disease or disorder.

The terms “elevated” or “increased” level of an indicator refer to the level of such indicator in the sample being higher in comparison to the level of such indicator in a reference or reference sample. E.g. a protein that is detectable in higher amounts in a fluid sample of one individual suffering from a given disease than in the same fluid sample of individuals not suffering from said disease, has an elevated level.

The term “biomarker” or “marker” or “biochemical marker” or “marker molecule” as used herein refers to a molecule to be used as a target for analyzing a patient's test sample. In one embodiment examples of such molecular targets are proteins or polypeptides. Proteins or polypeptides used as a marker in the present invention are contemplated to include naturally occurring fragments of said protein in particular, immunologically detectable fragments. Immunologically detectable fragments preferably comprise at least 6, 7, 8, 10, 12, 15 or 20 contiguous amino acids of said marker polypeptide. One of skilled in the art would recognize that proteins which are released by cells or present in the extracellular matrix may be damaged, e.g., during inflammation, and could become degraded or cleaved into such fragments. Certain markers are synthesized in an inactive form, which may be subsequently activated by proteolysis. As the skilled artisan will appreciate, proteins or fragments thereof may also be present as part of a complex. Such complex also may be used as a marker in the sense of the present invention. In addition, or in the alternative a marker polypeptide may carry a post-translational modification. Examples of posttranslational modifications amongst others are glycosylation, acylation, and/or phosphorylation. The term “biomarker” or “marker” as used herein refers generally to a molecule, including a gene, protein, carbohydrate structure, or glycolipid, metabolite, mRNA, miRNA, protein, DNA (cDNA or genomic DNA), DNA copy number, or an epigenetic change, e.g., increased, decreased, or altered DNA methylation (e.g., cytosine methylation, or CpG methylation, non-CpG methylations); histone modification (e.g., (de)acetylation, (de) methylation, (de) phosphorylation, ubiquitination, SUMOylation, ADP-ribosylation); altered nucleosome positioning, the expression or presence of which in or on a mammalian tissue or cell can be detected by standard methods (or methods disclosed herein) and which may be predictive, diagnostic and/or prognostic for a mammalian cell's or tissue's sensitivity to treatment regimes.

The term “sample” or “patient sample” as used herein refers to a biological sample obtained for the purpose of evaluation in vitro. In the methods of the present invention, the sample or patient sample preferably may comprise any body fluid. Test samples include blood, serum, plasma, urine, saliva, and synovial fluid. Preferred samples are whole blood, serum or plasma. As the skilled artisan will appreciate, any such assessment is made in vitro. The patient sample is discarded afterwards. The patient sample is solely used for the in vitro method of the invention and the material of the patient sample is not transferred back into the patient's body.

The term “patient” or “subject” herein is any single human subject eligible for treatment who is experiencing or has experienced one or more signs, symptoms, or other indicators of disease CCA. Intended to be included as a subject are any subjects involved in clinical research trials not showing any clinical sign of disease, or subjects involved in epidemiological studies, or subjects once used as controls.

An “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual is a human being.

The term “determining” the level of a biomarker, as used herein refers to the quantification of the biomarker, e.g. to determining or measuring the level of the biomarker in the sample, employing appropriate methods of detection described elsewhere herein.

In certain embodiments, the term “reference level” herein refers to a predetermined value. In this context “level” or “level value” encompasses the absolute amount, the relative amount or concentration as well as any value or parameter which correlates thereto or can be derived therefrom. As the skilled artisan will appreciate the reference level is predetermined and set to meet routine requirements in terms of e.g. specificity and/or sensitivity. These requirements can vary, e.g. from regulatory body to regulatory body. It may for example be that assay sensitivity or specificity, respectively, has to be set to certain limits, e.g. 80%, 90%, 95% or 98%, respectively. These requirements may also be defined in terms of positive or negative predictive values. Nonetheless, based on the teaching given in the present invention it will always be possible for a skilled artisan to arrive at the reference level meeting those requirements. In one embodiment the reference level is determined in reference samples from healthy individuals. The reference level in one embodiment has been predetermined in reference samples from the disease entity to which the patient belongs. In certain embodiments the reference level can e.g. be set to any percentage between 25% and 75% of the overall distribution of the values in a disease entity investigated. In other embodiments the reference level can e.g. be set to the median, tertiles or quartiles as determined from the overall distribution of the values in reference samples from a disease entity investigated. In one embodiment the reference level is set to the median value as determined from the overall distribution of the values in a disease entity investigated. The reference level may vary depending on various physiological parameters such as age, gender or subpopulation, as well as on the means used for the determination of the biomarker TIMP1 and optionally the combination of TIMP1 and MMP2 referred to herein. In one embodiment, the reference sample is from essentially the same type of cells, tissue, organ or body fluid source as the sample from the individual or patient subjected to the method of the invention, e.g. if according to the invention blood is used as a sample to determine the level of biomarker TIMP1 and optionally MMP2 in the individual, the reference level is also determined in blood or a part thereof.

In certain embodiments, the term “above the reference level or an increased level of the biomarker compared to the reference level” refers to a level of the biomarker in the sample from the individual or patient above the reference level or to an overall increase of 5%, 10%, 20%, 25%, 30%, 40%, 50%, 60%, 70%, 80%, 85%, 90%, 95%, 100% or greater, determined by the methods described herein, as compared to the reference level. In certain embodiments, the term increase refers to the increase in biomarker level in the sample from the individual or patient wherein, the increase is at least about 1.5-, 1.75-, 2-, 3-, 4-, 5-, 6-, 7-, 8-, 9-, 10-, 15-, 20-, 25-, 30-, 40-, 50-, 60-, 70-, 75-, 80-, 90-, or 100-fold higher as compared to the reference level, e.g. predetermined from a reference sample.

In certain embodiments, the term “decrease” or “below” herein refers to a level of the biomarker in the sample from the individual or patient below the reference level or to an overall reduction of 5%, 10%, 20%, 25%, 30%, 40%, 50%, 60%, 70%, 80%, 85%, 90%, 95%, 96%, 97%, 98%, 99% or greater, determined by the methods described herein, as compared to the reference level. In certain embodiments, the term decrease in biomarker level in the sample from the individual or patient wherein the decreased level is at most about 0.9-, 0.8-, 0.7-, 0.6-, 0.5-, 0.4-, 0.3-, 0.2-, 0.1-, 0.05-, or 0.01-fold of the reference level, e.g. predetermined from a reference sample, or lower.

The term “indicative for cholangiocarcinoma” or “indicates that the individual has CCA” is used to illustrate that a level of biomarker TIMP1 and optionally the combination of TIMP1 and MMP2 is very valuable but is not diagnostic without error. Not in all (100%) of the patients with disease CCA the level of biomarker TIMP1 is above the reference level and not in all healthy individuals the level of biomarker TIMP1 is lower than the reference level. As the skilled artisan will appreciate, in many diseases, no biochemical marker has 100% specificity and at the same time 100% sensitivity. In such case assessment e.g., with regard to the level of biomarker TIMP1 and optionally the combination with MMP2 in disease CCA is performed with a certain likelihood, e.g. at a given level of specificity or at a given level of sensitivity. The skilled artisan is fully familiar with the mathematical/statistical methods used to calculate specificity, sensitivity, positive predictive value, negative predictive value, reference value or total error. Any of these parameters can be calculated and used to obtain an indication of the presence or absence of disease CCA.

The term “comparing” as used herein refers to comparing the level of the biomarker in the sample from the individual or patient with the reference level of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer assisted. Thus, the comparison may be carried out by a computing device (e.g. of a system disclosed herein). The value of the measured or detected level of the biomarker in the sample from the individual or patient and the reference level can be, e.g., compared to each other and the said comparison can be automatically carried out by a computer program executing an algorithm for the comparison. The computer program carrying out the said evaluation will provide the desired assessment in a suitable output format. For a computer assisted comparison, the value of the determined amount may be compared to values corresponding to suitable references, which are stored in a database by a computer program. The computer program may further evaluate the result of the comparison, i.e. automatically provide the desired assessment in a suitable output format. Fora computer assisted comparison, the value of the determined amount may be compared to values corresponding to suitable references, which are stored in a database by a computer program. The computer program may further evaluate the result of the comparison, i.e. automatically provides the desired assessment in a suitable output format.

The expression “comparing the level determined in step (a) to the reference level of TIMP1” is merely used to further illustrate what is obvious to the skilled artisan anyway. The reference sample may be an internal or an external control sample. In one embodiment an internal reference sample is used, i.e. the marker level(s) is(are) assessed in the test sample as well as in one or more other sample(s) taken from the same subject to determine if there are any changes in the level(s) of said marker(s). In another embodiment an external reference sample is used. For an external reference sample the presence or amount of a marker in a sample derived from the individual is compared to its presence or amount in an individual known to stiffer from, or known to be at risk of, a given condition; or an individual known to be free of a given condition, i.e., “normal individual”. For example, a marker level in a patient sample can be compared to a level known to be associated with a specific course of disease in CCA. Usually the sample's marker level is directly or indirectly correlated with a diagnosis and the marker level is e.g. used to determine whether an individual is at risk for CCA. Alternatively, the sample's marker level can e.g. be compared to a marker level known to be associated with a response to therapy in CCA patients. Depending on the intended diagnostic use an appropriate reference sample is chosen and a control or reference value for the marker established therein. It will be appreciated by the skilled artisan that such reference sample in one embodiment is obtained from a reference population that is age-matched and free of confounding diseases. As also clear to the skilled artisan, the absolute marker values established in a reference sample will be dependent on the assay used. Preferably, samples from 100 well-characterized individuals from the appropriate reference population are used to establish a reference value. Also preferred the reference population may be chosen to consist of 20, 30, 50, 200, 500 or 1000 individuals. Healthy individuals represent a preferred reference population for establishing a control or reference value.

The phrase “providing an assessment” as used herein refers to using the information or data generated relating to the level or presence of TIMP1 and optionally MMP2 in a sample of a patient to assess CCA in the patient. The information or data may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a assessment of the level of TIMP1 and optionally in combination with MMP2 to an established reference level of TIMP1 and MMP2, respectively. In some embodiments, the information or data includes an indication that TIMP1 and optionally MMP2 is present or absent in the sample. In some embodiments, the information or data includes an indication that the patient is assessed with CCA.

The term “antibody and fragments thereof” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. In particular, the antibody is used herein as a “binding agent”. The term “binding agent” refers to a molecule that comprises a binding moiety which specifically binds the corresponding target biomarker TIMP1 and/or MMP2 molecule. Examples of “binding agent” are a nucleic acid probe, nucleic acid primer, DNA molecule, RNA molecule, aptamer, antibody, antibody fragment, peptide, peptide nucleic acid (ANA) or chemical compound. The antibody or fragments thereof has thus a specific binding affinity for TIMP1 and MMP2, respectively.

The term “specific binding” or “specifically binds” refers to a binding reaction wherein binding pair molecules exhibit a binding to each other under conditions where they do not significantly bind to other molecules.

The term “specific binding” or “specifically binds”, when referring to a protein or peptide as an antibody or binding agent, refers to a binding reaction wherein a binding agent binds to the corresponding target molecule with an affinity of at least 10⁻⁷ M. The term “specific binding” or “specifically bind” preferably refers to an affinity of at least 10⁻⁸ M or even more preferred of at least 10⁻⁹ M for its target molecule. The term “specific” or “specifically” is used to indicate that other molecules present in the sample do not significantly bind to the binding agent specific for the target molecule. Preferably, the level of binding to a molecule other than the target molecule results in a binding affinity which is only 10% or less, more preferably only 5% or less of the affinity to the target molecule.

The term “specific binding” or “specifically binds”, when referring to a nucleic acid as a binding agent, refers to a hybridization reaction wherein a binding agent or a probe contains a hybridizing region exactly or substantially complementary to the target sequence of interest. A hybridization assay carried out using the binding agent or probe under sufficiently stringent hybridization conditions enables the selective detection of the specific target sequence. The hybridizing region is preferably from about 10 to about 35 nucleotides in length, more preferably from about 15 to about 35 nucleotides in length. The use of modified bases or base analogues which affect the hybridization stability, which are well known in the art, may enable the use of shorter or longer probes with comparable stability. A binding agent or a probe can either consist entirely of the hybridizing region or can contain additional features which allow for the detection or immobilization of the probe, but which do not significantly alter the hybridization characteristics of the hybridizing region.

The term “specific binding” or “specifically binds”, when referring to a nucleic acid aptamer as a binding agent, refers to a binding reaction wherein a nucleic acid aptamer binds to the corresponding target molecule with an affinity in the low nM to pM range.

The term “detectable label” is an attachment of a specific tag to an antibody to aid in detection of an antibody. Numerous labels (also referred to as dyes) are available which can be generally grouped into the following categories, all of them together and each of them representing embodiments according the present disclosure:

### (a) Fluorescent Dyes

Fluorescent dyes are e.g. described by Briggs et al. “Synthesis of Functionalized Fluorescent Dyes and Their Coupling to Amines and Amino Acids,” J. Chem. Soc., Perkin-Trans. 1 (1997) 1051-1058).

Fluorescent labels or fluorophores include rare earth chelates (europium chelates), fluorescein type labels including FITC, 5-carboxytluorescein, 6-carboxy fluorescein; rhodamine type labels including TAMRA; dansyl; Lissamine; cyanines; phycoerythrins; Texas Red; and analogs thereof. The fluorescent labels can be conjugated to an aldehyde group comprised in target molecule using the techniques disclosed herein. Fluorescent dyes and fluorescent label reagents include those which are commercially available from Invitrogen/Molecular Probes (Eugene, Oregon, USA) and Pierce Biotechnology, Inc. (Rockford, Ill.).

### (b) Luminescent Dyes

Luminescent dyes or labels can be further subcategorized into chemiluminescent and electrochemiluminescent dyes.

The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439).

The labels of major relevance used as electrochemiluminescent labels are the Ruthenium- and the Iridium-based electrochemiluminescent complexes, respectively. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)₃]²⁻ (which releases a photon at ˜620 nm) regenerating with TPA (Tripropylamine) in liquid phase or liquid-solid interface are used as ECL-labels. Recently also Iridium-based ECL-labels have been described (WO2012/107419A1).

(c) Radioactive Labels Make Use of Radioisotopes (Radionuclides), Such as ³H, ¹¹C, ¹⁴C, ¹⁸F, ³²P, ³⁵S, ⁶⁴Cu, ⁶⁸Gn, ⁸⁶Y, ⁸⁹Zr, ⁹⁹TC, ¹¹¹In, ¹²³I, ¹²⁴I, ¹²⁵I, ¹³¹I, ¹³³Xe, ¹⁷⁷Lu, ²¹¹At, or ¹³¹Bi.
(d) Metal-chelate complexes suitable as labels for imaging and therapeutic purposes are well-known in the art (US 2010/0111856; U.S. Pat. Nos. 5,342,606; 5,428,155; 5,316,757; 5,480,990; 5,462,725; 5,428,139; 5,385,893; 5,739,294; 5,750,660; 5,834,456; Hnatowich et al, J. Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990), Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, et al, J. Nucl. Med. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals 18 (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

The term “signal from the detectable label of the antibody or fragment thereof” herein is used in the sense of an intensity signal obtained from the detectable label of the antibody or fragment thereof in a sample. The signal can be determined computer assisted. The signal can be carried out by a computing device.

The term “quantified signal calculated” means here and in the following that, as standard and routine, TIMP1 and optionally MMP2 is quantified and/or measured via a calibration curve.

The term “isolated” in connection with antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatmann al., J. Chromatogr. B 848:79-87 (2007).

TIMP1 (Tissue Inhibitor of Metalloproteinase 1) is a 184 amino acid sialoglycoprotein with a molecular weight of 28.5 kDa (see e.g. Murphy et al Biochem J. 1981, 195, 167-170) which inhibits metalloproteinases like interstitial collagenase MMP1 or stromelysin or gelatinase B. In the understanding of the current invention the term TIMP-1 encompasses a protein with significant structural homology to human TIMP1 inhibiting the proteolytic activity of metalloproteinases. The presence of human TIMP1 can be detected by using antibodies that specifically detect epitopes of TIMP1. TIMP1 may also be determined by detection of related nucleic acids such as the corresponding mRNA. The TIMP1 in the sense of the present invention can be characterized by the sequence given in SEQ ID NO: 1 or a homologous sequence thereof. In particularly, a homologous sequence has a amino acid sequence identity of at least 60%, 70%, 80%, 90%, 91%, 92%, 93%, 94%, 95%, 96%, 97%, 98% or 99%.

Matrix metalloproteinase-2 (MMP2), also called gelatinase A, is a 66 kDa zinc- and calcium-binding proteinase that is synthesized as an inactive 72 kD a precursor. MMP2 is synthesized by a variety of cells, including vascular smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP2 is usually found in plasma in complex with TIMP2, its physiological regulator (Murawaki, Y. et al., J. Hepatol. 30:1090-1098, 1999). The normal plasma concentration of MMP2 is <˜550 ng/ml (8 nM). MMP2 expression is elevated in vascular smooth muscle cells within atherosclerotic lesions, and it may be released into the bloodstream in cases of plaque instability (Kai, H. et al., J. Am. Coll. Gardiol. 32:368-372. 1998). Furthermore, MMP2 has been implicated as a contributor to plaque instability and rupture (Shah, P. K. et al., Circulation 92:1565-1569, 1995). Serum MMP2 concentrations were elevated inpatients with stable angina, unstable angina, and AMI, with elevations being significantly greater in unstable angina and AMI than in stable angina (Kai, H. et al., J. Am. Coll. Cardiol. 32:368-372, 1998). There was no change in the serum MMP2 concentration in individuals with stable angina following a treadmill exercise test (Kai, H. et al., J. Am. Coll. Cardiol. 32:368-372, 1998). Serum and plasma MMP2 is elevated in patients with gastric cancer, hepatocellular carcinoma, liver cirrhosis, urothelial carcinoma, rheumatoid arthritis, and lung cancer (Murawaki, Y., et al., J. Hepatol. 30:1090-1098, 1999; Endo, K., et al., Anticancer Res. 17:2253-2258, 1997; Gohji, K. et al., Cancer 78:2379-2387, 1996; Gruber, B. L. et al., Clin. Immunol. Immunopathol. 78:161-171, 1996; Garbisa, S. et al., Cancer Res. 52:4548-4549, 1992). Furthermore, MMP2 may also be translated from the platelet cytosol to the extracellular space during platelet aggregation (Sawicki, G. et al., Thromb. Haemost. 80:836-839, 1998). MMP2 was elevated on admission in the serum of individuals with unstable angina and AMI, with maximum levels approaching 1.5 μg/ml (25 nM) (Kai, H. et al., J. Am. Coll. Cardiol. 32:368-372, 1998). The serum MMP-2 concentration peaked 1-3 days after onset in both unstable angina and AMI, and started to return to normal after 1 week (Kai, H. et al., J. Am. Coll. Cardiol. 32:368-372, 1998).

The MMP2 in the sense of the present invention can be characterized by the sequence given in SEQ ID NO: 2 or a homologous sequence thereof. In particularly, a homologous sequence has a amino acid sequence identity of at least 60%, 70%, 80%, 90%, 91%, 92%, 93%, 94%, 95%, 96%, 97%, 98% or 99%.

The CA 19-9 (carbohydrate Antigen 19-9) values measured are defined by the use of the monoclonal antibody 1116-NS-19-9. The 1116-NS-19-9-reactive determinant in serum is mainly expressed on a mucin-like protein that contains a high number of CA19-9 epitopes (Magnani J. L., Arch. Biochem. Biophys. 426 (2004) 122-131). 3-7% of the population have the Lewis a-negative/b-negative blood group configuration and are unable to express the mucin with the reactive determinant CA 19-9. This must be taken into account when interpreting the findings. CA19-9 containing mucins are expressed in fetal gastric, intestinal and pancreatic epithelia. Low concentrations can also be found in adult tissue in the liver, lungs, and pancreas (Stieber, P. and Fateh-Moghadam, A., Boeringer Mannheim, Cat. No. 1536869 (engl), 1320947 (dtsch). ISBN 3-926725-07-9 dtsch/engl, Juergen Hartmann Verlag, Marloffstein-Rathsberg (1993); Herlyn. NI., et al., J. Clin. Immunol. 2 (1982) 135-140).

CEA (carcinoembryonic antigen) is a monomeric glycoprotein (molecular weight approx. 180.000 dalton) with a variable carbohydrate component of approx. 45-60% (Gold P. and Freedman S. O., J. Exp Med 121 (1965) 439-462). CEA, like AFP, belongs to the group of carcinofetal antigens that are produced during the embryonic and fetal period. The CEA gene family consists of about 17 active genes in two subgroups. The first group contains CEA and the Non-specific Cross-reacting Antigens (NCA); the second group contains the Pregnancy-Specific Glycoproteins (PSG). CEA is mainly found in the fetal gastrointestinal tract and in fetal serum. It also occurs in slight quantities in intestinal, pancreatic, and hepatic tissue of healthy adults. The formation of CEA is repressed after birth, and accordingly serum CEA values are hardly measurable in healthy adults. High CEA concentrations are frequently found in cases of colorectal adenocarcinoma (Stieber, P. and Fateh-Moghadam, A., supra). Slight to moderate CEA elevations (rarely >10 ng/mL) occur in 20-50% of benign diseases of the intestine, the pancreas, the liver, and the lungs (e.g. liver cirrhosis, chronic hepatitis, pancreatitis, ulcerative colitis, Crohn's Disease, emphysema (Stieber P., and Fateh-Moghadam A., supra). Smokers also have elevated CEA values. The main indication for CEA determinations is therapy management and the follow-up of patients with colorectal carcinoma. CEA determinations are not recommended for cancer-screening in the general population. CEA concentrations within the normal range do not exclude the possible presence of a malignant disease. The antibodies in assay manufactured by Roche Diagnostics react with CEA and (as with almost all CEA detection methods) with the meconium antigen (NCA2). Cross-reactivity with NCA1 is 0.7% (Hammarstrom S., et al., Cancer Res. 49 (1989) 4852-4858; and Bonner O. P., Tumor Biol. 12 (1991) 9-15).

### EMBODIMENTS

In a first aspect, the present invention relates to an in vitro method for assessing cholangiocarcinoma in a patient sample, comprising:
a) determining the level of tissue inhibitor of metalloproteinase-1 (TIMP1) in the patient sample, wherein the patient sample is selected from a group consisting of serum, plasma and whole blood sample from an individual,
b) comparing the level of TIMP1 determined in step (a) with a reference level of TIMP1, and
c) assessing cholangiocarcinoma in the patient sample by comparing the level determined in step (a) to the reference level of TIMP1, wherein an increased level of TIMP1 compared to the reference level of TIMP1 is indicative for cholangiocarcinoma in the patient sample.

The inventors of the present invention have surprisingly been able to demonstrate that the marker TIMP1 is useful in the assessment of CCA. Due to the uncertainties of classifying the various stages of CCA, it may well be that the TIMP1 may become one of the pivotal criteria in the assessment of patients with CCA in the future. The method of the present invention is suitable for the assessment of CCA. Increased levels, for example concentrations, of TIMP1 in a sample as compared to normal controls have been found to be indicative of CCA.

In embodiments of the first aspect of the present invention, the method comprises the step (a) of determining, step (b) of comparing and step (c) of assessing CCA. In particular, the method is done by the following order: step (a), followed by step (b) and followed by step (c).

In embodiments of the first aspect of the present invention, the level of tissue inhibitor of metalloproteinase-1 (TIMP1) in the patient sample is determined. The patient sample is selected from a group consisting of serum, plasma and whole blood sample from an individual. In particular, the patient sample is serum. The term “serum” as used herein is the clear liquid part of the blood hat can be separated from clotted blood. The term “plasma” as used herein is the clear liquid part of blood which contains the blood cells. Serum differs from plasma, the liquid portion of normal unclotted blood containing the red and white cells and platelets. It is the clot that makes the difference between serum and plasma. The term “whole blood” as used herein contains all components of blood, for examples white and red blood cells, platelets, and plasma. An increased level of TIMP1 compared to the reference level of TIMP1 is indicative for cholangiocarcinoma in the patient sample.

In embodiments of the first aspect of the present invention, the level of TIMP1 in the patient sample is determined by a sandwich type immunoassay (step (a)). The sandwich type immunoassay is known to a person skilled in the art. In such assay, a first specific binding agent is used to capture TIMP1 on the one side and a second specific binding agent, which is labelled to be directly or indirectly detectable, is used on the other side. The specific binding agents used in a sandwich-type assay format may be antibodies specifically directed against TIMP1. On the one hand, the detection may be carried out by using different capturing and labelled antibodies, i.e. antibodies which recognize different epitopes on the TIMP1. On the other hand, a sandwich-type assay may also be carried out with a capture and labelling antibody which is directed against the same epitope of TIMP1. In this embodiment, only di- and multimeric forms of TIMP1 may be detected. In an embodiment an antibody to TIMP1 is used in a qualitative (TIMP1 present or absent) or quantitative (amount of TIMP1 is determined) immunoassay. In particular, the level of TIMP1 in the patient sample is determined by an Elecsys-assay. An Elecsys-assays is known to person skilled in art and is therefore not explained in detail at this point.

In embodiments of the first aspect of the present invention, the level of TIMP1 in the patient sample is determined by a competitive immunoassay or enzyme-linked immunosorbent assay (ELISA).

For determination of TIMP1 the sample obtained from an individual is incubated in vitro with the specific binding agent for TIMP1 under conditions appropriate for formation of a binding agent TIMP1 complex. Such conditions need not be specified, since the skilled artisan without any inventive effort can easily identify such appropriate incubation conditions. The amount of binding agent TIMP1 complex is determined and used in the assessment of CCA. As the skilled artisan will appreciate there are numerous methods to determine the amount of the specific binding agent TIMP1 complex all described in detail in relevant textbooks (cf, e.g., Tijssen, P., supra, or Diamandis, E. P., and Christopoulos, T. K. (eds.), Immunoassay, Academic Press, Boston (1996)).

Immunoassays are well known to the skilled artisan. Methods for carrying out such assays as well as practical applications and procedures are summarized in related textbooks. Examples of related textbooks are Tijssen, P., Preparation of enzyme-antibody or other enzyme-macromolecule conjugates, In: Practice and theory of enzyme immunoassays, PP. 221-278, Burdon, R. H. and v. Knippenberg, P. H. (eds.), Elsevier, Amsterdam (1990), and various volumes of Colowick, S. P., and Caplan, N. O., (eds.), Methods in Enzymology, Academic Press, dealing with immunological detection methods, especially volumes 70, 73, 74, 84, 92 and 121.

In embodiments of the first aspect of the present invention, the marker TIMP1 is specifically determined in vitro from a liquid sample by use of a specific binding agent.

In embodiments of the first aspect of the present invention, a specific binding agent is, e.g., a receptor for the TIMP1, a lectin binding to TIMP1, an antibody to TIMP1, peptidebodies to TIMP1, bispecific dual binders or bispecific antibody formats. A specific binding agent has at least an affinity of 10⁷ l/mol for its corresponding target molecule. The specific binding agent preferably has an affinity of 10⁸ l/mol or also preferred of 10⁹ l/mol for its target molecule.

As the skilled artisan will appreciate the term specific is used to indicate that other biomolecules present in the sample do not significantly bind to the binding agent specific for the TIMP1 sequence of SEQ ID NO: 1. Preferably, the level of binding to a biomolecule other than the target molecule results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. Examples of specific binding agents are peptides, peptide mimetics, aptamers, spiegelmers, darpins, ankyrin repeat proteins, Kunitz type domains, antibodies, single domain antibodies, (see: Hey T. et al., Trends Biotechnol. 23 (2005) 514-30 522) and monovalent fragments of antibodies.

For the achievements as disclosed in the present invention antibodies from various sources may be used. Standard protocols for obtaining antibodies can be as well used as modern alternative methods. Alternative methods for generation of antibodies comprise amongst others the use of synthetic or recombinant peptides, representing a clinically relevant epitope of ASC for immunization. Alternatively, DNA immunization also known as DNA vaccination may be used. Clearly monoclonal antibodies or polyclonal antibodies from different species, e.g., rabbits, sheep, goats, rats or guinea pigs can be used. Since monoclonal antibodies can be produced in any amount required with constant properties, they represent ideal tools in development of an assay for clinical routine.

In certain preferred embodiments of the first aspect of the present invention, the specific binding agent is an antibody or fragment thereof. The fragment is preferably a monovalent antibody fragment, preferably a monovalent fragment derived from a monoclonal antibody.

As the skilled artisan will appreciate now, that TIMP1 has been identified as a marker, which is useful in the assessment of CCA. Various immunodiagnostic procedures may be used to reach data comparable to the achievements of the present invention.

In embodiments of the first aspect of the present invention step (a) comprises contacting, in vitro, a portion of the serum, plasma, or whole blood sample from the individual with an antibody or fragment thereof having specific binding affinity for TIMP1, thereby forming a complex between the antibody or fragment thereof and TIMP1 present in the patient sample, the antibody having a detectable label;
separating the complex formed in said step of contacting from antibody or fragment thereof not comprising the complex;
quantifying a signal from the detectable label of the antibody or fragment thereof comprising the complex formed in said step of contacting, the signal being proportional to an amount of TIMP1 present in the patient sample of the individual, whereby a level of TIMP1 within the sample of the individual is based on the quantified signal calculated.

In embodiments of the first aspect of the present invention, step (b) comprises: comparing the calculated level value of TIMP1 within the patient sample of the individual determined in said step of quantifying to a reference level of TIMP1.

In embodiments of the first aspect of the present invention, step (c) comprises: providing an assessment of cholangiocarcinoma in the individual when the calculated level value of TIMP1 is greater than the reference level of TIMP1.

In embodiments of the first aspect of the present invention, the steps (a) to (c) mentioned in the last 3 paragraphs above can be combined with each other.

In embodiments of the first aspect of the present invention, antibody or fragment thereof is an non mammalian antibody. The antibody or fragment thereof is not isolated from a human being.

In embodiments of the first aspect of the present invention, a specific binding agent preferably is an antibody specifically binding to TIMP1.

In embodiments of the first aspect of the present invention, said antibody or fragment thereof is isolated from an immunized animal, wherein the animal is selected from the group consisting of mice, rabbit, sheep, chicken, goat and guinea pig.

In embodiments of the first aspect of the present invention, TIMP1, particularly soluble forms of TIMP1, are determined in vitro in an appropriate sample. Preferably, the sample is derived from a human subject, e.g. a CCA and/or HHC patient and/or a person in risk of CCA and/or a person suspected of having CCA and/or a person in risk of HHC and/or a person suspected of having HHC.

In embodiments of the first aspect of the present invention, the sample is obtained from a human subject at risk control. “At risk control” as referred to herein means liver disease or injury selected from a group consisting of cirrhosis, chronic viral hepatitis, alcohol excess, non-alcoholic steatohepatitis, diabetes, obesity, hepatobiliary flukes, primary sclerosing cholangitis (PSC), biliary tract cysts, hepatolithiasis, toxins, primary biliary cirrhosis (PBC), primary hemochromatosis and combinations thereof.

In embodiments of the first aspect of the present invention, the sample is obtained from a human subject at risk control. The risk control is selected from a group consisting of cirrhosis, chronic viral hepatitis, alcohol excess, non-alcoholic steatohepatitis, diabetes, obesity, hepatobiliary flukes, primary sclerosing cholangitis (PSC), biliary tract cysts, hepatolithiasis, toxins, primary biliary cirrhosis (PBC), primary hemochromatosis and combinations thereof. In particular, the sample is obtained from a human subject, which is not diagnosed with choledocholithiasis.

In embodiments of the first aspect of the present invention, the method is used to differentiate cholangiocarcinoma from at risk control, which is selected from a group consisting of cirrhosis, chronic viral hepatitis, alcohol excess, non-alcoholic steatohepatitis, diabetes, obesity, hepatobiliary flukes, primary sclerosing cholangitis (PSC), biliary tract cysts, hepatolithiasis, toxins, primary biliary cirrhosis (PBC), primary hemochromatosis and combinations thereof.

In embodiments of the first aspect of the present invention, the method is used to differentiate cholangiocarcinoma from hepatocellular carcinoma.

In embodiments of the first aspect of the present invention, values for TIMP1 as determined in a control group or a control population are used to establish a reference range. In a preferred embodiment a level of TIMP1 is considered as elevated if the value determined is above the 90%-percentile of the reference range. In further preferred embodiments a level of TIMP1 is considered as elevated if the value determined is above the 95%-percentile, the 96%-percentile, the 97%-percentile or the 97.5%-percentile of the reference range.

In embodiments of the first aspect of the present invention, a sample provided from a patient with already confirmed CCA in certain settings might be used as a positive control sample and preferably assayed in parallel with the sample to be investigated. In such setting a positive result for the marker TIMP1 in the positive control sample indicates that the testing procedure has worked on the technical level.

In embodiments of the first aspect of the present invention, the patient sample is based on a liquid or body fluid sample which is obtained from an individual and on the in vitro determination of TIMP1 in such sample. An “individual” as used herein refers to a single human or non-human organism. Thus, the methods and compositions described herein are applicable to both human and veterinary disease. Preferably, the individual, subject, or patient is a human being.

It is known to a person skilled in the art that the measurement results of step (a) according to the method(s) of the present invention will be compared to a reference level of TIMP1. Such reference level can be determined using a negative reference sample, a positive reference sample, or a mixed reference sample comprising one or more than one of these types of controls. A negative reference sample preferably will comprise a sample from healthy individuals or individuals with no diagnosis of CCA. A positive reference sample preferably will comprise a sample from a subject with the diagnosis of CCA. In embodiments of the first aspect of the present invention, positive reference sample or negative reference sample is obtained from patients, which are not diagnosed with choledocholithiasis.

In embodiments of the first aspect of the present invention, the reference sample is particularly a biological sample provided from a reference group of apparently healthy individuals for the purpose of evaluation in vitro or individuals at risk of developing CCA. The term “reference value” as used herein refers to a value established in a reference group of apparently healthy individuals or individuals, which do not suffer from CCA or individuals at risk of developing CCA.

In embodiments of the first aspect of the present invention, said step (c) is performed by a computing device.

In a second aspect, the present invention relates to the use of TIMP1 as a marker molecule in the in vitro assessment of cholangiocarcinoma in a serum, plasma or whole blood sample of an individual, wherein a level of TIMP1 above a reference level of TIMP1 is indicative for cholangiocarcinoma. All embodiments mentioned above for the first aspect and/or other aspects of the present invention apply to the second aspect of the present invention and vice versa.

In a third aspect, the present invention relates to the use of a marker combination comprising TIMP1 and MMP2 in the in vitro assessment of cholangiocarcinoma in a serum, plasma or whole blood sample of an individual, wherein the levels of TIMP1 and MMP2 are indicative for cholangiocarcinoma. All embodiments mentioned above for the first and/or second aspects and/or other aspects of the present invention apply to the third aspect of the present invention and vice versa.

In embodiments of the third aspect of the present invention, the marker combination consists of TIMP1 and MMP2. This can mean that no other marker is present for assessing CCA.

In a fourth aspect, the present invention relates to an in vitro method for assessing cholangiocarcinoma in a patient sample, comprising:
(a′) determining the level of tissue inhibitor of metalloproteinase-1 (TIMP1) in the patient sample, wherein the patient sample is selected from a group consisting of serum, plasma and whole blood sample from an individual,
(b′) determining the level of matrix metalloproteinase-2 (MMP2) for cholangiocarcinoma in the patient sample, and
(c′) assessing cholangiocarcinoma by comparing the determining results of steps (a′) and (b′), wherein the levels of TIMP1 and MMP2 are indicative for CCA.

All embodiments mentioned above for the first, second, third and/or other aspects of the present invention apply to the fourth aspect of the present invention and vice versa.

Preferably, embodiments mentioned for step (a) apply for step (a′).

In embodiments of the fourth aspect of the present invention, step (c′) comprises: assessing cholangiocarcinoma by comparing the determining results of steps (a′) and (b′), wherein the detected levels of TIMP1 and MMP2 are indicative for CCA.

In embodiments of the fourth aspect of the present invention, step (a′) comprises: contacting, in vitro, a portion of the serum, plasma, or whole blood sample from the individual with an antibody or fragment thereof having specific binding affinity for TIMP1, thereby forming a complex between the antibody or fragment thereof and TIMP1 present in the patient sample, the antibody having a detectable label;
separating the complex formed in said step of contacting from antibody or fragment thereof not comprising the complex; and
quantifying a signal from the detectable label of the antibody or fragment thereof comprising the complex formed in said step of contacting, the signal being proportional to an amount of TIMP1 present in the patient sample of the individual, whereby a level of TIMP1 within the sample of the individual is based on the quantified signal calculated.

In embodiments of the fourth aspect of the present invention, step (b′) comprises: contacting, in vitro, a portion of the serum, plasma, or whole blood sample from the individual with an antibody or fragment thereof having specific binding affinity for MMP2, thereby forming a complex between the antibody or fragment thereof and MMP2 present in the patient sample, the antibody having a detectable label;
separating the complex formed in said step of contacting from antibody or fragment thereof not comprising the complex; and
quantifying a signal from the detectable label of the antibody or fragment thereof comprising the complex formed in said step of contacting, the signal being proportional to an amount of MMP2 present in the patient sample of the individual, whereby a level of MMP2 within the sample of the individual is based on the quantified signal calculated.

In embodiments of the fourth aspect of the present invention, steps (a′) and (b′) mentioned above in the last two paragraphs can be combined with each other.

In embodiments of the fourth aspect of the present invention, step (c′) comprises: including the level of MMP2 determined in step (b′) and the level of TIMP1 determined in step (a′) into a statistical methodology to produce an output value that indicates whether the patient sample has cholangiocarcinoma or is at risk of developing cholangiocarcinoma.

In embodiments of the fourth aspect of the present invention, the statistical methodology used is, e.g. logistic regression.

In embodiments of the fourth aspect of the present invention, multivariate score is calculated.

Other methods, e.g. selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods) can also be used.

The performance of the results of the applied statistical methods used in accordance with the present invention can be best described by their receiver operating characteristics (ROC). The ROC curve addresses both the sensitivity (the number of true positives) and the specificity (the number of true negatives) of the test. Therefore, sensitivity and specificity values for a given biomarker or a combination of biomarkers are an indication of the performance of the test. For example, if a biomarker combination has a sensitivity and specificity value of 80%, out of 100 diseased patients, 80 will be correctly identified from the determination of the presence of the particular combination of biomarkers as positive for the disease, while out of 100 patients who do not have the disease 80 will accurately test negative for the disease.

A suitable statistical classification model, such as logistic regression, can be derived for a combination of biomarkers with or without clinical variables. Moreover, the logistic regression equation can be extended to include other (clinical) variables such as age and gender of the patient as well. In the same manner as described before, the ROC curve can be used to access the performance of the discrimination between patients and controls by the logistic regression model. Although a logistic regression equation is a common statistical procedure used in such cases and is preferred in the context of the current invention, other mathematical or statistical methods such as decision trees or machine learning procedures can also be used.

One convenient goal to quantify the diagnostic accuracy of a laboratory test is to express its performance by a single number. The most common global measure is the area under the curve (AUC) of the ROC plot. The area under the ROC curve is a measure of the probability that the perceived measurement will allow correct identification of a condition. Values typically range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0). In the context of the present invention, the two different conditions can be whether a patient has or does not have cancer like CCA.

In embodiments of the fourth aspect of the present invention, steps (a′), (b′) and (c′) mentioned above in the last three paragraphs can be combined with each other.

In embodiments of the fourth aspect of the present invention, each of the steps (a′) and (b′) comprises a sandwich type immunoassay.

In embodiments of the fourth aspect of the present invention, said step (c′) is performed by a computing device.

In embodiments of the fourth aspect of the present invention, the sample is obtained from a human subject at risk control. The risk control is selected from a group consisting of cirrhosis, chronic viral hepatitis, alcohol excess, non-alcoholic steatohepatitis, diabetes, obesity, hepatobiliary flukes, primary sclerosing cholangitis (PSC), biliary tract cysts, hepatolithiasis, toxins, primary biliary cirrhosis (PBC), primary hemochromatosis and combinations thereof. In particular, the sample is obtained from a human subject, which is not diagnosed with choledocholithiasis.

In embodiments of the fourth aspect of the present invention, the method is used to differentiate cholangiocarcinoma from at risk control, which is selected from a group consisting of cirrhosis, chronic viral hepatitis, alcohol excess, non-alcoholic steatohepatitis, diabetes, obesity, hepatobiliary flukes, primary sclerosing cholangitis (PSC), biliary tract cysts, hepatolithiasis, toxins, primary biliary cirrhosis (PBC), primary hemochromatosis and combinations thereof.

In embodiments of the fourth aspect of the present invention, the method is used to differentiate cholangiocarcinoma from hepatocellular carcinoma.

In embodiments of the fourth aspect of the present invention, a specific binding agent preferably is an antibody specifically binding to MMP2.

In a fifth aspect, the present invention relates to a kit for performing the method according to the first aspect and/or according to the fourth aspect of the present invention comprising reagents, which are required to determine the level of TIMP1 determined in step (a) or step (a′) and optionally to determine the level of MMP2 determined in step (b′). All embodiments mentioned above for the first, second, third, fourth and/or other aspects of the present invention apply to the fifth aspect of the present invention and vice versa.

In embodiments of the fifth aspect of the present invention, the kit comprises the reagent or reagents required to specifically determine the level of TIMP1 and the reagent or reagents required to determine or measure marker MMP2 that are used together in an CCA marker combination. Said kit comprises in an embodiment antibodies or fragments thereof specifically binding to TIMP1. In a further embodiment said antibody fragments in said kit are selected from the group consisting of Fab, Fab′, F(ab′)2, and Fv. In one embodiment the present invention relates to a kit comprising at least two antibodies or fragments thereof specifically binding to at least two non-overlapping epitopes comprised in the TIMP1 sequence of SEQ ID NO: 1. Preferably the at least two antibodies or fragments thereof comprised in a kit according to the present invention are monoclonal antibodies. Said kit further comprises in an embodiment a bio-chip on which the antibodies or fragments thereof are immobilized.

In embodiments of the fifth aspect of the present invention, the reagent for TIMP1 and/or MMP2 is a combination of one or more components, such as probes (for example, an antibody), controls, buffers, reagents (for example, conjugate and/or substrate) instructions, and the like, as disclosed herein.

Certain illustrative embodiments are as follows:
1. An in vitro method for assessing cholangiocarcinoma in a patient sample, comprising:
a) determining the level of tissue inhibitor of metalloproteinase-1 (TIMP1) in the patient sample, wherein the patient sample is selected from a group consisting of serum, plasma and whole blood sample from an individual,
b) comparing the level of TIMP1 determined in step (a) with a reference level of TIMP1, and
c) assessing cholangiocarcinoma in the patient sample by comparing the level determined in step (a) to the reference level of TIMP1, wherein an increased level of TIMP1 compared to the reference level of TIMP1 is indicative for cholangiocarcinoma in the patient sample.
2. The method according to embodiment 1,
wherein step (a) comprises: contacting, in vitro, a portion of the serum, plasma, or whole blood sample from the individual with an antibody or fragment thereof having specific binding affinity for TIMP1, thereby forming a complex between the antibody or fragment thereof and TIMP1 present in the patient sample, the antibody having a detectable label;
   separating the complex formed in said step of contacting from antibody or fragment thereof not comprising the complex;
   quantifying a signal from the detectable label of the antibody or fragment thereof comprising the complex formed in said step of contacting, the signal being proportional to an amount of TIMP1 present in the patient sample of the individual, whereby a level of TIMP1 within the sample of the individual is based on the quantified signal calculated; and/or
   wherein step (b) comprises: comparing the calculated level value of TIMP1 within the patient sample of the individual determined in said step of quantifying to a reference level of TIMP1; and/or
   wherein step (c) comprises: providing an assessment of cholangiocarcinoma in the individual when the calculated level value of TIMP1 is greater than the reference level of TIMP1.
3. The method according to embodiment 1 or 2, wherein said antibody or fragment thereof is isolated from an immunized animal, wherein the animal is selected from the group consisting of mice, rabbit, sheep, chicken, goat and guinea pig.
4. The method according to any one of the embodiments 1 to 3, wherein step (a) comprises a sandwich type immunoassay.
5. The method according to any one of the embodiments 1 to 4, wherein said step (c) is performed by a computing device.
6. Use of TIMP1 as a marker molecule in the in vitro assessment of cholangiocarcinoma in a serum, plasma or whole blood sample of an individual, wherein a level of TIMP1 above a reference level of TIMP1 is indicative for cholangiocarcinoma.
7. Use of a marker combination comprising TIMP1 and MMP2 in the in vitro assessment of cholangiocarcinoma in a serum, plasma or whole blood sample of an individual, wherein the levels of TIMP1 and MMP2 are indicative for cholangiocarcinoma.
8. An in vitro method for assessing cholangiocarcinoma in a patient sample, comprising:
(a′) determining the level of tissue inhibitor of metalloproteinase-1 (TIMP1) in the patient sample, wherein the patient sample is selected from a group consisting of serum, plasma and whole blood sample from an individual,
(b′) determining the level of matrix metalloproteinase-2 (MMP2) for cholangiocarcinoma in the patient sample, and
(c′) assessing cholangiocarcinoma by comparing the determining results of steps (a′) and (b′), wherein the levels of TIMP1 and MMP2 are indicative for CCA.
9. The method according to embodiment 8,
wherein step (a′) comprises: contacting, in vitro, a portion of the serum, plasma, or whole blood sample from the individual with an antibody or fragment thereof having specific binding affinity for TIMP1, thereby forming a complex between the antibody or fragment thereof and TIMP1 present in the patient sample, the antibody having a detectable label;
   separating the complex formed in said step of contacting from antibody or fragment thereof not comprising the complex;
   quantifying a signal from the detectable label of the antibody or fragment thereof comprising the complex formed in said step of contacting, the signal being proportional to an amount of TIMP1 present in the patient sample of the individual, whereby a level of TIMP1 within the sample of the individual is based on the quantified signal calculated; and/or
wherein step (b′) comprises: contacting, in vitro, a portion of the serum, plasma, or whole blood sample from the individual with an antibody or fragment thereof having specific binding affinity for MMP2, thereby forming a complex between the antibody or fragment thereof and MMP2 present in the patient sample, the antibody having a detectable label;
   separating the complex formed in said step of contacting from antibody or fragment thereof not comprising the complex;
   quantifying a signal from the detectable label of the antibody or fragment thereof comprising the complex formed in said step of contacting, the signal being proportional to an amount of MMP2 present in the patient sample of the individual, whereby a level of MMP2 within the sample of the individual is based on the quantified signal calculated.
10. The method according to embodiment 8 or 9, wherein step (c′) comprises:
   including the level of MMP2 determined in step (b′) and the level of TIMP1 determined in step (a′) into a statistical methodology to produce an output value that indicates whether the patient sample has cholangiocarcinoma or is at risk of developing cholangiocarcinoma.
11. The method according to any one of the embodiments 8 to 10, wherein each of the steps (a′) and (b)′) comprises a sandwich type immunoassay.
12. The method according to any one of the embodiments 8 to 11, wherein said step (c′) is performed by a computing device.
13. The method according to any one of the embodiments 1 to 5 or according to any one of the embodiments 8 to 12 to differentiate cholangiocarcinoma from hepatocellular carcinoma.
14. The method according to any one of the embodiments 1 to 5 or according to any one of the embodiments 8 to 12 to differentiate cholangiocarcinoma from at risk control, which is selected from a group consisting of cirrhosis, chronic viral hepatitis, alcohol excess, non-alcoholic steatohepatitis, diabetes, obesity, hepatobiliary flukes, primary sclerosing cholangitis (PSC), biliary tract cysts, hepatolithiasis, toxins, primary biliary cirrhosis (PBC), primary hemochromatosis and combinations thereof
15. A kit for performing the method according to any one of embodiments 1 to 5 or according to any one of the embodiments 8 to 12 comprising reagents, which are required to determine the level of TIMP1 determined in step (a) or step (a′) and optionally to determine the level of MMP2 determined in step (b′).

These embodiments are intended to be illustrative, and are not intended to limit the scope of the invention.

The following examples, sequence listing and figures are provided to aid the understanding of the present invention, the true scope of which is set forth in the appended claims. It is understood that modifications can be made in the procedures set forth without departing from the spirit of the invention.

All references cited in this specification are herewith incorporated by reference with respect to their entire disclosure content and the disclosure content specifically mentioned in this specification.

### DESCRIPTION OF THE SEQUENCES

SEQ ID NO: 1 shows the amino acid sequence of TIMP1 (sequence listing; Uniprot P01033, Version 214).

SEQ ID NO: 2 shows the amino acid sequence of MMP2 (sequence listing; Uniprot P08253, Version 235).

### EXAMPLES

The invention will be merely illustrated by the following Examples. The said Examples shall, whatsoever, not be construed in a manner limiting the scope of the invention.

### Example 1

### Study Population:

Clinical performance of the biomarkers CEA, CA19-9, TIMP-1 and MMP2 for diagnosis of cholangiocarcinoma was evaluated in sample panel composed of
55 CCA samples: 20 intrahepatic, 11 perihilar, 24 no information.
219 HCC samples.
632 controls representing patients at risk for development of CCA and HCC (cirrhosis, chronic viral hepatitis, alcoholic, and non-alcoholic steatohepatitis).

### Blood Serum Collection:

Serum samples were collected by the following institutions: Maharaj Nakorn Chiang Mai Hospital, Chiang Mai, Thailand; Siriraj Hospital, Bangkok, Thailand; Songklanagarind Hospital, HatYai Songkhla, Thailand; Srinagarind Hospital, KhonKaen, NCI Universitätsklinikum Heidelberg, Heidelberg, Germany and University Hospital Vall d'Hebron, Barcelona, Spain. The study was conducted in full conformance with the principles of the “Declaration of Helsinki” and with approval of the independent ethic committee (IEC). Serum samples were collected before treatment (surgery, Percutaneous Ethanol Injection (PEI), chemotherapy, radiotherapy) according to the appropriate Standard Operating Procedures (SOPs) and stored at <−70° C. until analysis. Repeated freezing and thawing was avoided.

### Serum Sample Preparation:

Serum samples were drawn into a serum tube and allowed to clot for at least 60 minutes up to 120 minutes at room temperature. After centrifugation (10 min, 2000 g), the supernatant was divided into 1 ml aliquots and frozen at −70° C. Before measurement, the samples were thawed, re-aliquoted into smaller volumes appropriate for prototype assays and reference assays and refrozen. Samples were thawed immediately before analysis. Therefore, each sample in the panel had only two freeze-thaw cycles before measurement.

### Example 2

### Roche Elecsys® Assays for Detection of Tumor Marker:

Elecsys® kits were acquired for the tumor markers CEA and CA19-9. All these markers were measured with Roche Elecsys® in vitro diagnostic. All assays were run according to the manufacturer's instructions (Roche Diagnostics GmbH, Mannheim, Germany). The concentrations measured by the instrument were used to calculate/generate the AUC data shown in Tables 1 and 5.

### Example 3

ELISA for the Measurement of the Level of TIMP1 and Optionally in Combination with the Level of MMP2 in Human Serum or Plasma Samples:

For measurement of TIMP-1 commercially available MTP ELISAs from R&D Systems Inc. (Minneapolis, USA) were used (Quantikine® ELISA, Human TIMP-1 Immunoassay, Catalog Numbers DTM100, STM100, PDTM100). Briefly, this assay employs the quantitative sandwich enzyme immunoassay technique. A monoclonal antibody specific for human TIMP-1 has been pre-coated onto a microplate. Standards and samples are pipetted into the wells and any TIMP-1 present is bound by the immobilized antibody. After washing away any unbound substances, an enzyme-linked polyclonal antibody specific for human TIMP-1 is added to the wells. Following a wash to remove any unbound antibody-enzyme reagent, a substrate solution is added to the wells and color develops in proportion to the amount of TIMP-1 bound in the initial step. The color development is stopped and the intensity of the color is measured.

For measurement of MMP2 commercially available MTP ELISAs from R&D Systems Inc. (Minneapolis, USA) were used (Quantikine® ELISA, Total MMP-2 Immunoassay, Catalog Numbers MMP200, SMMP200, PMMP200). Briefly, this assay employs the quantitative sandwich enzyme immunoassay technique. A monoclonal antibody specific for Total MMP-2 has been pre-coated onto a microplate. Standards and samples are pipetted into the wells and any MMP-2 present is bound by the immobilized antibody. After washing away any unbound substances, an enzyme-linked polyclonal antibody specific for Total MMP-2 is added to the wells. Following a wash to remove any unbound antibody-enzyme reagent, a substrate solution is added to the wells and color develops in proportion to the amount of Total MMP-2 bound in the initial step. The color development is stopped and the intensity of the color is measured.

### Example 4

Uni- and Multivariate Analysis were Performed to Identify Biomarker/Biomarker Panel for Best Differentiation of:
CCA from HCC.
CCA from at-risk controls.
CCA from HCC at-risk controls.

### Univariate Analysis

The performance of TIMP1 is evaluated based on its absolute measured value. All measured values from diseased and non-diseased included patients can be used as threshold to calculate the sensitivity and specificity at this specific value. Based on those combinations of sensitivities and specificities the ROC curve can be drawn and the AUC (the area under the ROC curve) can be calculated.

### Multivariate Analysis

The combination of the markers TIMP1 and MMP2 is evaluated. Preferably the values measured for markers of a marker combination TIMP1 and MMP2 are mathematically combined and the combined value is correlated to the underlying diagnostic question. Marker values may be combined by any appropriate state of the art mathematical method.

Preferably the method used in correlating the marker combination of the invention e.g. to the absence or presence of CCA is a Generalized Linear Model (i.e. Logistic Regression). However, other methods, e.g. selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods) can also be used.

Details relating to these statistical methods are found in the following references: Ruczinski, I., et al, J. of Computational and Graphical Statistics, 12 (2003) 475-511; Friedman, J. H., J. of the American Statistical Association 84 (1989) 165-175; Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. (1984) Classification and regression trees, California: Wadsworth; Breiman, L., Random Forests, Machine Learning, 45 (2001) 5-32; Pepe, M. S., The Statistical Evaluation of Medical Tests for Classification and Prediction, Oxford Statistical Science Series 28 (2003); and Duda, R. O., Hart, P. E., Stork, D. G., Pattern Classification, Wiley Interscience, 2nd Edition (2001).

It is a preferred embodiment of the invention to use an cut-off for the underlying combination of biological markers and to discriminate state A from state B, e.g. diseased from healthy. In this type of analysis the markers are no longer independent but form a marker panel or marker combination.

Accuracy of a diagnostic method is best described by its receiver-operating characteristics (ROC) (see especially Zweig, M. H., and Campbell, G. Clin. Chem. 39 (1993) 561-577). The ROC graph is a plot of all of the sensitivity/specificity pairs resulting from continuously varying the decision threshold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1−specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1−specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1−specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One preferred way to quantify the diagnostic accuracy of a laboratory test is to express its performance by a single number. Such an overall parameter e.g. is the so-called “total error” or alternatively the “area under the curve=AUC”. The most common global measure is the area under the ROC plot. By convention, this area is always >0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

A binary logistic regression model including the log-transformed TIMP1 and MMP2 as independent and the diagnosis as dependent variable is used. With log-transformation it is meant that both variables are separately transformed by log 10 (TIMP1+0.1) and log 10 (MMP2+0.1). The prediction of the logistic regression model, which are in this case the log odds (probability on logit-scale) are used as the multivariate score. The multivariate score is handled as a continuous covariate based on which sensitivity and specificity could be derived for each single threshold.

### Example 5: TIMP1 as a Marker to Differentiate Human CCA Vs. HCC, CCA Vs. Risk Controls and CCA Vs. Risk Controls+HCC, Respectively

The serum concentration of TIMP1 in the CCA samples is evaluated in comparison to HCC samples and at-risk controls samples. The risk-control samples represent patients, which are at risk for development of CCA and HCC (cirrhosis, chronic viral hepatitis, alcoholic, and non-alcoholic steatohepatitis, 8 biliary cirrhosis and 4 PSC.

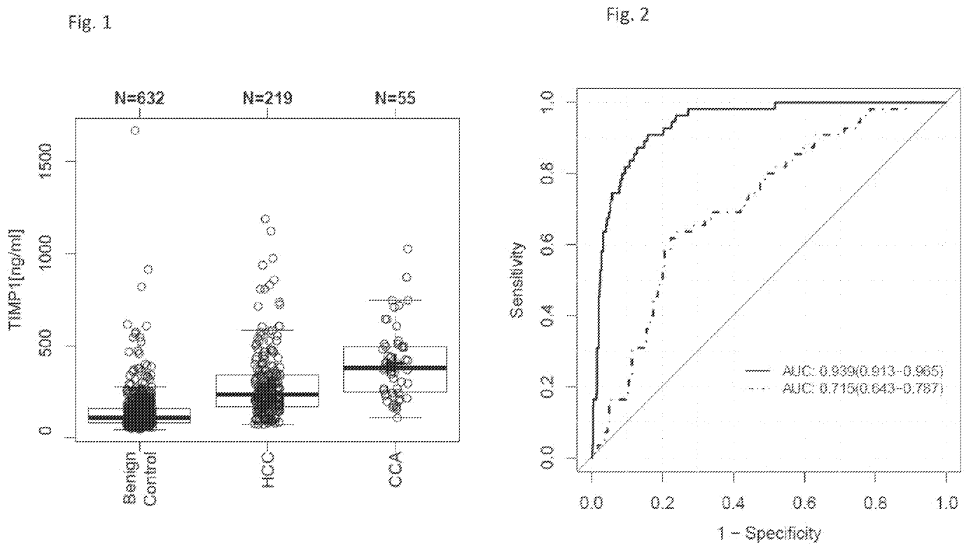

FIG. 1 shows the box plot of the distribution of the marker level values of TIMP1 (concentration values of TIMP1 in ng/ml) according to CCA (CCC) of the 55 samples, HCC of 219 HCC samples and at-risk controls of 632 samples.

FIG. 2 shows the plot of the receiver operator characteristics (ROC-plot, univariant analysis) of CCA vs. at-risk controls samples with an AUC of 0.939. Additionally, FIG. 2 shows the plot of the receiver operator characteristics (ROC-plot) of CCA vs. HCC samples with an AUC of 0.715.

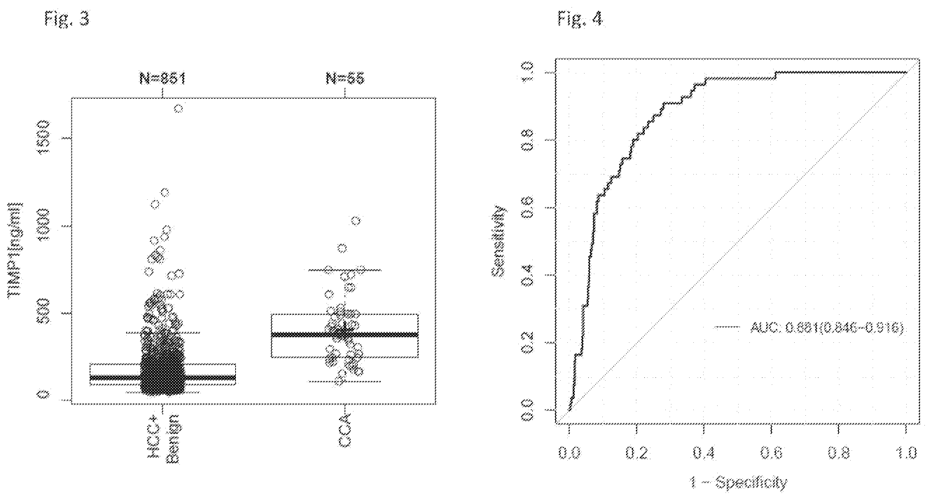

FIG. 3 shows the boxplot of the distribution of the determined TIMP1 level values (concentration values of TIMP1 in ng/ml) according to CCA (CCC) of the 55 samples and HCC with at-risk controls of 851 samples.

FIG. 4 shows the plot of the receiver operator characteristics (ROC-plot, univariant analysis) of TIMP1 in differentiating CCA from HCC+at-risk controls with an AUC of 0.881.

In univariate model, clinical performance of TIMP1 is significantly better as of reference markers CA19-9 and CEA, respectively. TIMP1 levels or concentrations in serum or plasma of CCA patients is significantly increased in comparison to HCC patients. Even better differentiation of concentration of TIMP1 is observed between CCA patients and control group consisting of patients at risk for development of CCA and HCC (cirrhosis, chronic viral hepatitis, alcoholic, and non-alcoholic steatohepatitis).

Clinical performance of TIMP-1 for diagnosis of CCA exceeds those of reference markers CA 19-9 and CEA (see Table 1):
a) for differential diagnosis CCA vs. HCC: TIMP-1 AUC 0.715, CA19-9 AUC 0.672, CEA AUC 0.615.
b) for diagnosis CCA vs. at-risk controls inclusive cirrhosis, chronic viral hepatitis, alcoholic, and non-alcoholic steatohepatitis: TIMP-1 AUC 0.939, CA19-9 AUC 0.784, CEA AUC 0.485.
c) for diagnosis CCA vs. HCC and at-risk controls: TIMP-1 AUC 0.881, CA19-9 AUC 0.755, CEA AUC 0.518.

TABLE 1
AUC values of the biomarkers (BM) TIMP-1, CA19-9 and CEA

| | | CCA vs at-risk | CCA vs HCC + at- |
|---|---|---|---|
| Biomarker | CCA vs HCC AUC | controls AUC | risk controls AUC |
| TIMP1 | 0.715 | 0.939 | 0.881 |
| CA 19-9 | 0,672 | 0.784 | 0.755 |
| CEA | 0.615 | 0.485 | 0.518 |

The inventors have surprisingly found out that this novel blood biomarker TIMP1 is highly sensitive and specific for the non-invasive diagnosis of CCA. Metalloproteinase inhibitor I TIMP-1 (Uniprot P01033) was not previously assessed in this context of cholangiocarcinoma.

### Example 6

Marker Combinations: TIMP1 and MMP2 as a Marker Combination to Differentiate Human CCA Vs. HCC, CCA Vs. Risk Controls and CCA Vs. Risk Controls+HCC Respectively:

In the multivariate analysis combination of TIMP-1 and MMP2 was selected as a best model outperforming all other marker combinations, based on the AUC of each combination of two biomarkers, which were included in the analyses.

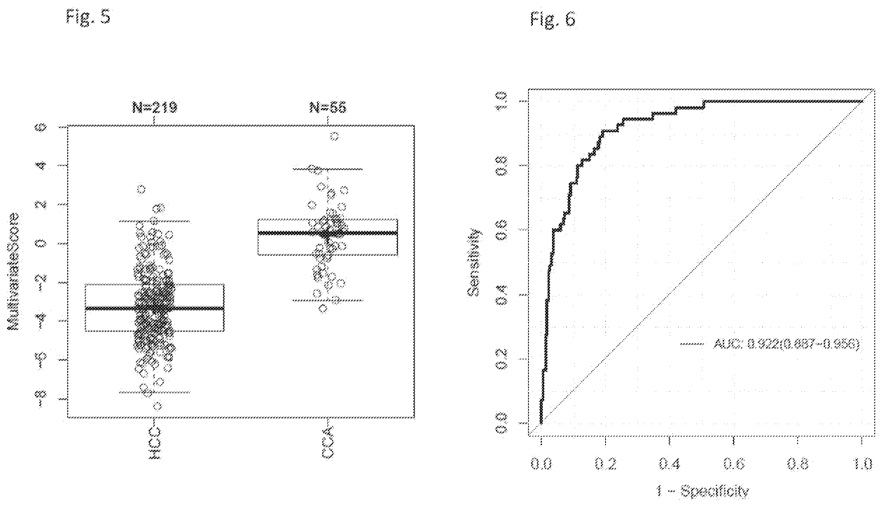

FIG. 5 shows the boxplot of the distribution of the multivariate score based on TIMP1 and MMP2 according to CCA (CCC) of 55 samples and HCC of 219 samples.

FIG. 6 shows the plot of the receiver operator characteristics (ROC-plot, multivariate analysis) of the marker combination TIMP1 and MMP2 in differentiating CCA from HCC with an AUC of 0.922.

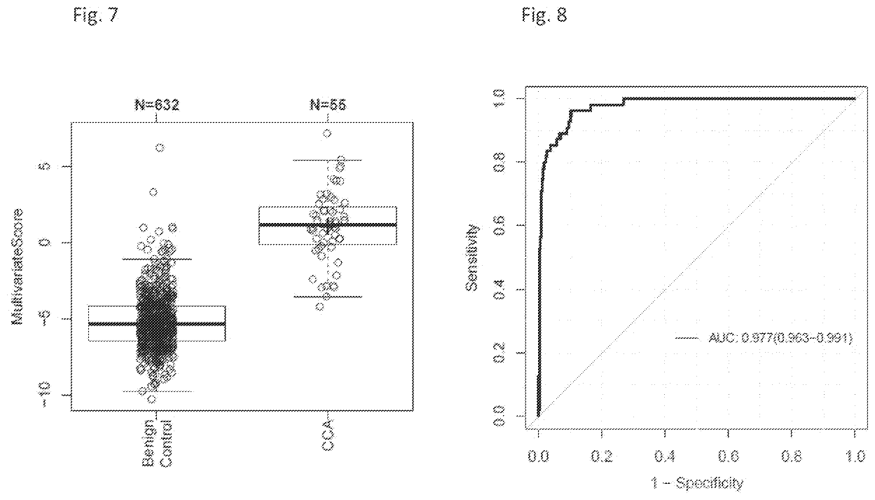

FIG. 7 shows the boxplot of the distribution of the multivariate score based on TIMP1 and MMP2 according to CCA (CCC) of the 55 samples and at-risk controls of 632 samples.

FIG. 8 shows the plot of the receiver operator characteristics (ROC-plot, multivariate analysis) of the marker combination TIMP1 and MMP2 in differentiating CCA from at-risk controls with an AUC of 0.977.

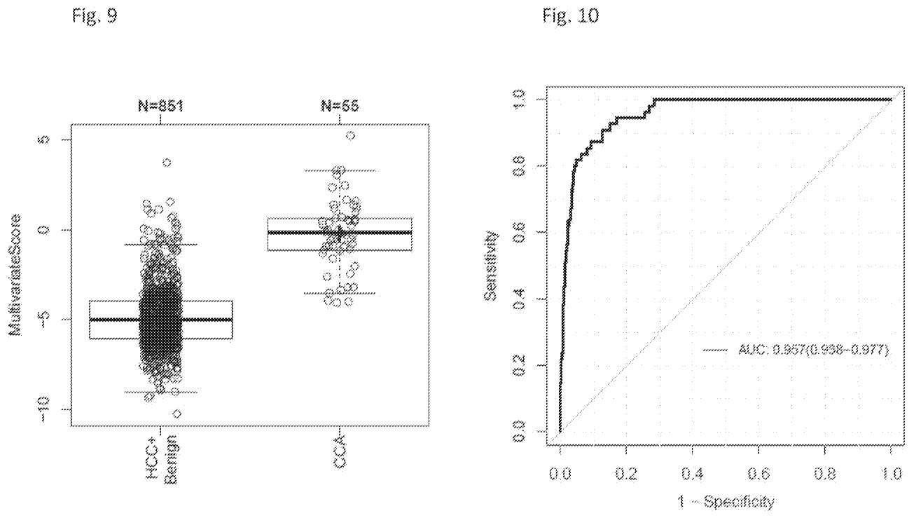

FIG. 9 shows the boxplot of the distribution of the marker combination TIMP1 and MMP2 according to CCA (CCC) of 55 samples and at-risk controls of 851 samples.

FIG. 10 shows the plot of the receiver operator characteristics (ROC-plot, multivariate analysis) of the marker combination TIMP1 and MMP2 in differentiating CCA vs. HCC+at-risk controls with an AUC of 0.957.

Clinical performance of TIMP-1 and MMP2 marker combination for diagnosis of CCA:
a) for differential diagnosis CCA vs. HCC: AUC 0.922.
b) for diagnosis CCA vs. at-risk controls inclusive cirrhosis, chronic viral hepatitis, alcoholic, and non-alcoholic steatohepatitis: AUC 0.977.
c) for diagnosis CCA vs. HCC and at-risk controls:AUC 0.957.

The determined AUC values are summarized in Table 5.

TABLE 5
AUC values for the biomarker (BM) combination of TIMPI and MMP2

| BM | CCA vs | CCA vs at-risk | CCA vs HCC + at- |
|---|---|---|---|
| combination | HCC AUC | controls AUC | risk controls AUC |
| TIMP1 + MMP2 | 0.922 | 0.977 | 0.957 |

1. An in vitro method for assessing cholangiocarcinoma in a patient sample, comprising:
a) determining the level of tissue inhibitor of metalloproteinase-1 (TIMP1) in the patient sample, wherein the patient sample is selected from the group consisting of serum, plasma and whole blood sample from an individual,
b) comparing the level of TIMP1 determined in step (a) with a reference level of TIMP1, and
c) assessing cholangiocarcinoma in the patient sample by comparing the level determined in step (a) to the reference level of TIMP1, wherein an increased level of TIMP1 compared to the reference level of TIMP1 is indicative for cholangiocarcinoma in the patient sample.

2. The method according to claim 1,
wherein step (a) comprises: contacting, in vitro, a portion of the serum, plasma, or whole blood sample from the individual with an antibody or fragment thereof having specific binding affinity for TIMP1, thereby forming a complex between the antibody or fragment thereof and TIMP1 present in the patient sample, the antibody having a detectable label;
separating the complex formed in said step of contacting from antibody or fragment thereof not comprising the complex; and
quantifying a signal from the detectable label of the antibody or fragment thereof comprising the complex formed in said step of contacting, the signal being proportional to an amount of TIMP1 present in the patient sample of the individual, whereby a level of TIMP1 within the sample of the individual is based on the quantified signal calculated.

3. The method according to claim 1, wherein said antibody or fragment thereof is isolated from an immunized animal, wherein the animal is selected from the group consisting of mice, rabbit, sheep, chicken, goat and guinea pig.

4. The method according to claim 1, wherein step (a) comprises a sandwich type immunoassay.

5. The method according to claim 1, wherein said step (c) is performed by a computing device.

6. An in vitro method for assessing cholangiocarcinoma in a patient sample, comprising:
(a′) determining the level of tissue inhibitor of metalloproteinase-1 (TIMP1) in the patient sample, wherein the patient sample is selected from the group consisting of serum, plasma and whole blood sample from an individual,
(b′) determining the level of matrix metalloproteinase-2 (MMP2) for cholangiocarcinoma in the patient sample, and
(c′) assessing cholangiocarcinoma by comparing the determined results of steps (a′) and (b′), wherein the levels of TIMP1 and MMP2 are indicative for CCA.

7. The method according to claim 6,
wherein step (a′) comprises: contacting, in vitro, a portion of the serum, plasma, or whole blood sample from the individual with an antibody or fragment thereof having specific binding affinity for TIMP1, thereby forming a complex between the antibody or fragment thereof and TIMP1 present in the patient sample, the antibody having a detectable label;
separating the complex formed in said step of contacting from antibody or fragment thereof not comprising the complex; and
quantifying a signal from the detectable label of the antibody or fragment thereof comprising the complex formed in said step of contacting, the signal being proportional to an amount of TIMP1 present in the patient sample of the individual, whereby a level of TIMP1 within the sample of the individual is based on the quantified signal calculated.

8. The method according to claim 6, wherein step (c′) comprises:
including the level of MMP2 determined in step (b′) and the level of TIMP1 determined in step (a′) into a statistical methodology to produce an output value that indicates whether the patient sample has cholangiocarcinoma or is at risk of developing cholangiocarcinoma.

9. The method according to claim 6, wherein each of the steps (a′) and (b′) comprises a sandwich type immunoassay.

10. The method according to claim 6, wherein said step (c′) is performed by a computing device.

11. The method according to claim 1, wherein the method differentiates cholangiocarcinoma from hepatocellular carcinoma.

12. The method according to claim 1, wherein the method differentiates cholangiocarcinoma from at risk control, which is selected from a group consisting of cirrhosis, chronic viral hepatitis, alcohol excess, non-alcoholic steatohepatitis, diabetes, obesity, hepatobiliary flukes, primary sclerosing cholangitis (PSC), biliary tract cysts, hepatolithiasis, toxins, primary biliary cirrhosis (PBC), primary hemochromatosis and combinations thereof.

13. A kit for performing the method according to claim 6 comprising antibodies or fragments thereof, which are required to determine a level of TIMP1 and a level of MMP2 in the sample.

14. The method according to claim 2, wherein step (b) comprises comparing the calculated level value of TIMP1 within the patient sample of the individual determined in said step of quantifying to a reference level of TIMP1.

15. The method according to claim 2, wherein step (c) comprises providing an assessment of cholangiocarcinoma in the individual when the calculated level value of TIMP1 is greater than the reference level of TIMP1.

16. The method according to claim 8, wherein the statistical methodology is selected from linear analysis, quadratic analysis, regularized discriminant analysis, kernel methods, nonparametric methods, logistic regression, CART, random forest methods, and boosting methods.

17. The method according to claim 16, wherein the statistical methodology is logistic regression including clinical variables for age and gender of the patient.

18. The method according to claim 16, wherein the statistical methodology is logistic regression, and wherein a multivariate score is calculated.

19. The method according to claim 7,
wherein step (b′) comprises contacting, in vitro, a portion of the serum, plasma, or whole blood sample from the individual with an antibody or fragment thereof having specific binding affinity for MMP2, thereby forming a complex between the antibody or fragment thereof and MMP2 present in the patient sample, the antibody having a detectable label;
separating the complex formed in said step of contacting from antibody or fragment thereof not comprising the complex; and
quantifying a signal from the detectable label of the antibody or fragment thereof comprising the complex formed in said step of contacting, the signal being proportional to an amount of MMP2 present in the patient sample of the individual, whereby a level of MMP2 within the sample of the individual is based on the quantified signal calculated.

20. A kit for performing the method according to claim 1 comprising an antibody or fragment thereof, which is required to determine a level of TIMP1 in the sample.
